(19) United States
(12) Patent Application Publication
(10) Pub. No.: US 2012/0321573 A1
(43) Pub. Date: Dec. 20, 2012

(54) COMPOSITIONS AND METHODS OF PROPHYLAXIS FOR CONTACT DERMATITIS

(75) Inventors: Jeffrey M. Karp, Brookline, MA (US); Praveen Kumar Vemula, Cambridge, MA (US); Richard Rox Anderson, Boston, MA (US)

(73) Assignees: THE GENERAL HOSPITAL CORPORATION, Boston, MA (US); THE BRIGHAM AND WOMEN'S HOSPITAL, INC., Boston, MA (US)

(21) Appl. No.: 13/499,820

(22) PCT Filed: Oct. 1, 2010
(86) PCT No.: PCT/US10/51125

§ 371 Date: Jul. 24, 2012

Related U.S. Application Data
(60) Provisional application No. 61/248,023, filed on Oct. 2, 2009.

(51) Int. Cl. A61K 33/42; C01B 25/32; A61K 33/10; A61P 17/16; A61P 29/00; C12N 5/071; A61P 17/04; A61P 37/06; A61L 2/16; A61L 2/22; A61L 2/23; C01F 11/18; A61P 31/00

(52) U.S. Cl. 424/59; 423/430; 423/305; 424/687; 424/602; 422/28; 435/375; 428/402

(57) ABSTRACT

The present invention relates to compositions and methods for inhibiting metal exposure to tissues.

### RELATED APPLICATIONS

This application claims benefit under 35 U.S.C. §119(e) of the U.S. Provisional Application No. 61/248,023 filed Oct. 2, 2009, the content of which is incorporated herein by reference in its entirety.

### TECHNICAL FIELD

This invention relates to compositions and methods of inhibiting metal induced contact dermatitis.

### BACKGROUND OF THE INVENTION

Metal induced contact dermatitis is a common reaction to minute amounts of metal ions or particles that directly contact the skin, and the number of people affected is increasing dramatically. See for example Marks, et al., Arch Dermatol 2000, 136(2):272-3; Rietschel, et al., Dermatitis 2008, 19(1):16-9; and Jacob, et al., J Am Acad Dermatol 2009, 60(6):1067-9. Nickel, for example, can evoke allergic dermatitis via transfusion, inhalation, and oral intake (e.g. through dental casting alloys (Marks, et al., Arch Dermatol 2000 136(2):272-3 and Hidaka, et al., J Biomed Mater Res 1994, 28(2):175-80) or via orthopedic and other implants (Dou, et al., Contact Dermatitis 2003, 48(3):126-9; Raison-Peyron, et al., Contact Dermatitis 2005, 53(4):222-5; and Nosbaum, et al., Contact Dermatitis 2008, 59(5):319-20). However, the most prevalent form is topical nickel induced contact dermatitis from jewelry and personal metal objects such as cell phones (Luo, et al. Cmaj 2008, 178(1):23-4), wristwatches, buttons (Suneja, et al., Dermatitis 2007, 18(4):208-11), snaps (Heim, et al., Contact Dermatitis 2009, 60(2):100-5) and currency coins (Nestle, et al., Nature 2002, 419(6903):132 and Staton, et al., Br J Dermatol 2006, 154(4): 658-64). Infants as young as 6 months old have been reported to be sensitized to nickel (Bruckner, et al., Pediatrics 2000, 105(1):e3). Many governments around the world have imposed restrictions to minimize nickel exposure. Although several prophylaxis agents have been developed to reduce nickel penetration through the skin, they fail due to their inefficiency, complexity, short-lasting activity and potential side-effects. See, for example, Memon, et al., J Am Acad Dermatol 1994, 30(4):560-5; Menne T, & Kaaber K. Contact Dermatitis 1978, 4(5):289-90; Wohrl, et al., Contact Dermatitis 2001, 44(4):224-8; Pantini, et al., Int J Cosmet Sci 1990, 12(6):273-9; Kaaber, et al., Contact Dermatitis 1979, 5(4):221-8; Martindale W. The Extra Pharmacopoeia: Pharmaceutical Press; 1993; Christensen O B, & Kristensen M. Contact Dermatitis 1982, 8(1):59-63; and Hopfer, et al., Res Commun Chem Pathol Pharmacol 1987, 55(1):101-9. As a result, existing prophylaxis agents are ineffective for prolonged exposure to a nickel source, and often may promote other forms of dermatitis or toxicity causing side-effects including neurotoxicity (Kaaber, et al., Contact Dermatitis 1979, 5(4):221-8), lassitude (Martindale W. The Extra Pharmacopoeia: Pharmaceutical Press; 1993), hepatotoxicity (Christensen O B, & Kristensen M. Contact Dermatitis 1982, 8(1):59-63), and accumulation within the brain (Hopfer, et al., Res Commun Chem Pathol Pharmacol 1987, 55(1):101-9). Furthermore, chelating prophylaxis agents may penetrate through the skin and escape into the blood stream causing toxicity. See, for example, Gawkrodger, et al., Contact Dermatitis 1995, 32(5):257-65 and Kauffman, et al., Pediatrics 1990, 86(5):797-798.

Thus, there is need for prophylaxis for nickel induced contact dermatitis (NCD) that can capture nickel ions efficiently, is non-toxic and does not absorb through the skin, even when the barrier is reduced as a result of allergic contact dermatitis. In addition, an ideal prophylaxis for NCD should be dispersible in an emollient or vehicle that is suitable for topical applications and should capture nickel ions under a wide range of pH conditions (e.g. in the presence of sweat).

### SUMMARY OF THE INVENTION

In one aspect, the invention features a composition for inhibiting metal exposure to tissue, the composition comprising a capturing agent. In some embodiments, the capturing agent is a non-covalently crosslinked capturing agent. In some embodiments, the capturing agent cannot transverse through tissue or does so negligibly. In some embodiments, the capturing agent is a nanoparticle. In some embodiments, the capturing agent is an inorganic capturing agent. In some embodiments, the composition further comprises at least one emollient and/or immuno-suppressing agent. In some embodiments, the capturing agent comprises at least one of a silicate, a carbonate, a sulfate, a phosphate, a citrate or an oxalate.

In another aspect, the invention provides a method of inhibiting metal exposure to tissue, the method comprising applying to the tissue a composition comprising at least one capturing agent.

In yet another aspect, the invention provides a method of inhibiting metal release from an object, the method comprising coating the object with a composition described herein.

In yet still another aspect, the invention provides a method of inhibiting activity of matrix metalloproteinases (MMP), the method comprising applying to the tissue a composition described herein.

### BRIEF DESCRIPTION OF THE DRAWINGS

FIG. 1 shows prophylaxis efficiency as a function of nanoparticle size. (a) Nickel quantities that are released into the artificial sweat at different time points from coated and uncoated nickel wires (the coated wires contained different sized CaCO₃-nanoparticles). The data shows that particles under ˜500 nm are most efficient nickel chelators. In all cases, values are average of three independent experiments and all standard deviations were <5% of the average values. (b) XPS spectra of nickel sequestered CaP and CaCO₃-particles, and NiSO₄ salt. Arrows show shoulder peaks.

FIG. 2 shows a schematic of nickel permeation experiment with and without prophylaxis coating on the full-thickness pig skin.

FIG. 3 shows the Energy-dispersive X-ray diffraction (EDX) spectra of nickel bound calcium carbonate particles. EDX spectra of CaCO₃-particles that were suspended in NiSO₄ solution for 48 hrs, then separated via centrifugation and washed with deionized water. Appearance of characteristic peaks of nickel 0.85 (Ni-Lα) and 7.47 (Ni-Kα) (blue arrows) suggesting that nickel ions that were present in solution were being captured by CaCO₃-particles. This is further evidenced by EDX spectra of native CaCO₃-particles (inset) where peaks corresponding to nickel were absent.

FIG. 4 shows histographs of CaCO₃ particle sizes. Size distribution of particles has been quantified by dynamic light scattering (ZEN 3690, Malvern Instruments, Inc.) for samples (a) 70; (b) 500; (c) 1000 and (d) 3000 nm population.

FIG. 5 shows EDX spectra of uncoated (FIG. 6A) and CaCO₃-particles coated (FIG. 6B) nickel wires.

FIG. 6 is a schematic representation of preparation of nickel wires coated with CaCO₃ or CaP particles. For preparation of coated nickel wires, in step-1 either CaCO₃ or CaP-particles were suspended in aqueous solution, bare nickel wires were incubated in for 1 hr, subsequently wires were removed and dipped in aqueous solution (step-2). Rinsing wires in aqueous solution (step-3) removes excess particles on the wire. Upon drying under in the air produced particles coated metal wires.

FIG. 7 shows a schematic representation of the metal diffusion through the skin experiment.

### DETAILED DESCRIPTION OF THE INVENTION

In one aspect, the invention features a composition for capturing and/or binding irritants to inhibit topical irritant induced contact dermatitis. As used herein, the term “irritant” means a substance that initiates an immunological event on contact with a tissue. The immunological event may lead to inflammation at the contact site. It is to be understood that, “to inhibit” includes preventing, reducing, lowering, stopping and/or reversing irritant induced contact dermatitis. Additionally, substances that degrade one or more components of the stratum corneum are also considered to be irritants for the purposes of the invention described herein

In some embodiments, the tissue is skin or mucosa.

In some embodiments, the irritant is a metal, preferably a soft or hard Lewis acid. In a preferred embodiment, metal is a soft Lewis acid and selected from the group consisting of Cu⁺, Ag⁺, Au⁺, Tl⁺, Hg⁺, Cs⁺, Zn²⁺, Ni²⁺, Pd²⁺, Cd²⁺, Pt²⁺, Hg²⁺, Tl³⁺, or metal atoms with zero oxidation state. Without wishing to be bound by theory, in general, soft acids/bases have less charge (lower charge state) and larger radius, whereas hard acids/bases have a high charge and smaller radius.

In another preferred embodiment, the metal is a hard Lewis acid and selected from the group consisting of Cr³⁺, Co³⁺, Fe³⁺, La³⁺, In³⁺, Ga³⁺, Sr³⁺, Al³⁺, or metal atoms with zero oxidation state.

In one preferred embodiment, the irritant is nickel.

In some embodiments, the irritant is not a metal, i.e. a non-metal irritant. Exemplary non-metal irritants include, but are not limited to, cytokines (such as interleukin-1a, IL-1(3 and IL-8), eicosanoids (such as PGE2 and LTB4), enzymes (such as kinase, tryptase, phospholipase, and glycosidase), proteases, lipases, glycosidases, bile acids, endotoxins, superantigens (such as those produced by the bacterium Staphylococcus aureus including staphylococcal enterotoxins A, B, and Toxic shock syndrome toxin-1), bacterial by-products, poison ivy, latex, grass, weeds, trees, animal products, and dust.

Due to the large surface area to volume ratio, nanoparticles have been used as chelating agents for a variety of applications. See, for example, White, et al., J Hazard Mater 2009, 161(2-3):848-53 and Huhtinen, et al., Anal Chem 2005, 77(8):2643-8. Accordingly, in some embodiments, the capturing agent is a nanoparticle based capturing agent. By “nanoparticle based capturing agent” is meant a nanoparticle which comprises a capturing agent. Without wishing to be bound by theory, said capturing agent can be present on the outer and/or inner surface of the nanoparticle. In some embodiments, the capturing agent is in the core of the nanoparticle. In some embodiments, the nanoparticle can be composed entirely of capturing agent, e.g., the capturing agent itself is the nanoparticle. Nanoparticle based capturing agents can be a nanoshell, nanocage, core-shell particle, nano-rod, nano-wire, nanocube, hollow nanosphere, aggregate of nanoparticles, or a combination thereof.

A non-zero concentration of a chelating agent can be included in the core of the nanoparticle based capturing agents. By non-zero concentration is meant that at least one chelating agent is present within the core of the nanoparticle. The chelating agent can be included within the core by covalent linkage to the particle or by non-covalent interactions.

Exemplary chelating agents include ethylenediamine tetra acetic acid (EDTA), terpyridine, triethylenetetramine, dimethyl glyoxime, oxalic acid and tartaric acid.

In some embodiments, the composition comprises an encapsulating agent that can encapsulate the capturing agent, e.g., nanoparticle based capturing agent. The encapsulating agents can be used to apply the capturing agent to tissue surface so that the capturing agent will remain on the surface of the tissue. The encapsulation agent can be a natural or synthetic polymer or a hydrogel. As used herein, the term “hydrogel” refers to a network of polymer chains that are water-insoluble. Hydrogels can comprise natural or synthetic polymers and can be a colloidal gel in which water is the dispersion medium. Exemplary hydrogels are described in Menger, F. M. & Caran, K. L. (2000) J. Am. Chem. Soc. 122, 11679-11691; Sreenivasachary, N. & Lehn, J.-M. (2005) Proc. Natl. Acad. Sci. USA 102, 5938-5943; Makarević, J., Jokić, M., Percić, B., Tomi\ddot{s}ić, V., Krojić-Prodić, B. & \ddot{Z}inić, M. (2001) Chem. Eur. J. 7, 3328-3341; Oda, R., Huc, I. & Candau, S. J. (1998) Angew. Chem. Int. Ed. 37, 2689-2691; Estroff, L. A. & Hamilton, A. D. (2000) Angew. Chem. Int. Ed. 39, 3447-3450; Kobayashi, H., Friggeri, A., Koumoto, K., Amaike, M., Shinkai, S. & Reinhoudt, D. N. (2002) Org. Lett. 4, 1423-1426; Luboradzki, R., Gronwald, O., Ikeda, M., Shinkai, S. & Reinhoudt, D. N. (2000) Tetrahedron 56, 9595-9599; Jung, J. H., John, G., Masuda, M., Yoshida, K., Shinkai, S. & Shimizu, T. (2001) Langmuir 17, 7229-7232; and Wang, G. & Hamilton, A. D. (2003) Chem. Commun. 310-311, content of all of which is herein incorporated by reference.

In some embodiments, the capturing agent can not traverse through tissue. A capturing agent is said to not transverse through a tissue, when upon application to surface of the tissue, the capturing agent does not penetrate into the tissue or does so negligibly over a given period of time, e.g., within 1, 5, 10, 20, 30, 60, 120, or 240 minutes. In some embodiments, the capturing agent penetrates into the tissue less than 1, 2, 3, 4, 5, 10, 50, 100, or 1000 nm from the outer surface of the tissue over a given period of time. In some other embodiments, the capturing agent penetrates into the tissue less than 1, 2, 3, 4, 5, 10, 50, 100, or 1000 μm from the surface of the tissue over a given period of time. In some embodiments, the capturing agent does not transverse a cell membrane.

In some embodiments, the capturing agent is a non-covalently crosslinked capturing agent. By “non-covalently crosslinked capturing agent” is meant a capturing agent wherein the different parts of the capturing agent are non-covalently bound to each other. The non-covalent binding can be through hydrogen bonds, van der Waals interactions, ionic interactions, non-ionic interactions, hydrophobic interactions, or a combination thereof. In some embodiments, the capturing agent comprises both crosslinked and non-crosslinked components, e.g. some parts are covalently linked with each other while some other parts are non-covalently bound.

In some embodiments, capturing agent has a crystalline structure, e.g. capturing agent is a crystalline compound.

In some embodiments, the composition comprises a capturing agent, wherein the capturing agent has a surface area greater than or equal to 1, 10, 20, or 50 m²/g.

In some embodiments, the composition comprises an insoluble capturing agent. As used here in, “insoluble capturing agent” refers to a capturing agent that does not dissolve in a liquid of interest or does so negligibly, or dissolves upon external stimuli. In some embodiments, the insoluble capturing agent has a water content greater than 10%, 50%, or 75%.

As used herein, the term “capturing agent” means a substance or material with an affinity for an irritant such that the irritant covalently or non-covalently binds to the capturing agent when in the proximity of the capturing agent. In some embodiments, the affinity for the irritant is high, rapid, and/or irreversible. Without wishing to be bound by a theory, irritant interaction with the capturing agent precludes or inhibits the ability of such irritant to cause contact dermatitis.

In some embodiments, the capturing agent is an inorganic capturing agent. As used here, the term “inorganic capturing agent” refers to a capturing agent that is of mineral origin. In some embodiments, the capturing agent comprises both inorganic and organic components. As used herein, the term, “inorganic” refers to compounds that are of mineral origin, and the term “organic” refers compounds that are of biological origin. It is to be understood that, biological origin does not mean that the compound itself is synthesized biologically. When a capturing agent comprises both inorganic and organic components, such components may or may not be covalently linked to each other.

Upon capture of irritant molecules, the capturing agent can under go physical and/or chemical changes. For example, the capturing agent can become insoluble, e.g., form a precipitate, upon binding with an irritant. In some embodiments, capturing agent releases an ion, upon binding with an irritant. In some embodiments, capturing agent releases calcium ions upon binding with an irritant.

As used herein, the term “capturing” means the binding of an irritant to a capturing agent. Capturing can be achieved using many well-known affinity-ligand systems, such as adsorbent clays, calcium carbonate, talc, silica, titanium dioxide (TiO₂), apatite, e.g., hydroxyapatite, alumina, deactivated alumina, aluminum silicate, MgSO₄, calcium silicate, activated carbon, pearl starch, calcium sulfate, antibodies, aptamer nucleic acids, ion-exchange materials, cyclodextrins, lectins, Lewis acid/base materials, activated charcoal, glass microspheres, diatomaceous earth, derivatives and combinations of the above.

Without wishing to be bound by theory, capturing agents inhibit metal exposure to tissue by inhibiting entry of said metal into the tissue. The capturing agents can act as a barrier to entry for the irritants or capturing agent can bind and sequester said irritant. It is understood that these two distinct modes of actions are not mutually exclusive and can be combined.

In some embodiments, the capturing agent is a chelating agent. As used herein, the term “chelating agent” refers to a molecule having unshared electron pairs available for donation to a metal ion. The metal ion is in this way coordinated by the chelating agent. A chelating agent can be a bidentate chelating agent, tridentate chelating agent, or quadradentate chelating agent. The terms, “bidentate chelating agent”, “tridentate chelating agent”, and “quadradentate chelating agent” refer to chelating agents having, respectively, two, three, and four electron pairs readily available for simultaneous donation to a metal ion coordinated by the chelating agent.

Metal complexing chelators can include monodentate and polydentate chelators. Metal complexing chelators include tetradentate metal chelators which can be macrocyclic and have a combination of four nitrogen and/or sulphur metal-coordinating atoms. Multidentate chelators can also incorporate other metal-coordinating atoms such as oxygen and phosphorous in various combinations. The metal binding complexing moiety can also include “3+1” chelators.

In some embodiments, the capturing agent is not a chelating agent as the term is defined herein.

In some embodiments, the capturing agent is an adsorbent agent. As used herein, the term “adsorbent agent” refers to a molecule that is capable of adsorbing a metal ion primarily by physical adsorption. Exemplary adsorbents include, but are not limited to, carbon aerogel, activated carbon, coal ash, wood saw dust (e.g., maple saw dust), macroalgae, kaolinite and activated slug. Other adsorbent agents amenable to the present invention are described in U.S. Pat. No. 5,185,313, content of which are herein incorporated by reference.

In some embodiments, the capturing agent is a dendritic molecule. As used herein the term “dendritic” refers to a hyperbranched structure, including multiple generations of branching, which has a high degree of regularity in branching, which can approach the regularity in branching of a true dendrimer but which may typically include some irregularities in branching. The term dendritic also includes the so called “hyper comb-branched” structures. Exemplary dendritic molecules amenable to the present invention are disclosed in U.S. Pat. Nos. 6,020,457; 7,261,876 and 6,995,234, contents of which are herein incorporated by reference.

In one embodiment, the capturing agent is a particle. As used herein, a “particle” is a small object that behaves as a whole unit in terms of its physical and chemical properties. The term particle, as used herein does not constitute individual molecules. Exemplary particles include nanoparticles and microparticles.

A non-zero concentration of a chelating agent can be included in the core of the capturing agent particles. By non-zero concentration is meant that at least one chelating agent is present within the core of the capturing agent particle. The chelating agent can be included within the core by covalent linkage to the particle or by non-covalent interactions.

As used herein, the term “nanoparticle” includes compounds that have at least one dimension in the 1-1000 nm range. The term nanoparticles also include compounds that are of mineral origin. The term “mineral” means a naturally occurring solid that has a characteristic chemical composition, a highly ordered atomic structure, and specific physical properties. There are ˜4,000 currently known minerals. Minerals are usually classified according to chemical composition. In many cases such classification is based on the anion group. Exemplary anion groups include various silicates, carbonate, sulfates, halides, oxides, sulfides, and phosphates. The organic mineral class includes biogenic substance in which geological processes have been a part of the genesis or origin of the existing compound. Anions of the organic class minerals include various oxalates, mellitates, citrates, cyanates, acetates, formates and hydrocarbons.

As one of skill in the art is aware, particles can comprise a variety of shapes, including, but not limited to, non-symmetrical, irregular, spherical, rod-like, elongated, star-shape or a combination thereof. Nanoparticles can be nanospheres, hollow nanospheres, nanorods, nanofibers, nanocups, nanoshells, nanocages, core-shell particles, nano-wires or nanocubes thereof.

Nanoparticle capturing agents can be synthesized using methods well known in the art and easily available to one of skill in the art. Exemplary nanoparticle methods include, but are not limited to, attrition, pyrolysis, inert-gas condensation, and sol-gel (Chemical Solution Deposition).

In certain embodiments, the particle is a Janus particle. As used herein, a “Janus particle” is a particle that is composed of at least two physically or chemically differing surfaces. A Janus particle can be composed of two fused hemispheres, wherein each hemisphere is made from a different chemical entity. Janus particles can be made of any combination of two or more different capturing agents and thus exhibit two or more different irritant capturing properties. The different capturing properties can be to capture different irritants, capture similar irritants at different rates, different capturing capacity, or a combination thereof. Without wishing to be bound by theory, Janus particle have two or more different prophylaxis properties. Janus particles can be synthesized using methods known in the art for example as described in U.S. Patent Application Publication Nos. 200/0105972 and 2008/0001116, contents of which are herein incorporated by reference.

In certain embodiments, the nanoparticle is a porous nanoparticle. Porous nanoparticle will have pores of sufficient size to allow entry of irritant into the interior of the nanoparticle. Without wishing to be bound by theory, entry of the irritant into the pores leads to capture of irritant.

In some embodiments, the capturing agent is a polymer or polymer based. As used herein, the term “polymer” refers broadly to a material made up of a chain of identical, repeated base units. Exemplary metal chelating polymers are described in U.S. Pat. Nos. 6,087,452; 5,286,887; 3,715,335; 5,770,637 and 4,190,709, contents of which are herein incorporated by reference.

In some embodiments, the capturing agent is a polymeric particle. In some embodiments the polymeric particle has one dimension in the range of 10-5000 nm. As used herein, the term “polymeric particle” refers to natural or synthetic particle comprising a polymer.

In some embodiments, the capturing agent is a degradable particle. As used herein, the term “degradable” means that the capturing agent particle is capable of decomposing into smaller molecules. Such decomposition can be by various chemical and/or physical mechanisms.

In certain embodiments, the capturing agent is a metal particle. In some embodiments, the metal particle is silver particle, gold particle, copper particle, platinum particle, palladium, titanium dioxide particle, magnetic particle or a quantum dot. In some embodiments, the metal particle has one dimension in the range of 10-200 nm.

In certain embodiments, capturing agent and/or particle is coated, derivatized or linked with a ligand. In some embodiments, the ligand is a derivative of ethylenediamine tetra acetic acid (EDTA), terpyridine, triethylenetetramine, dimethyl glyoxime, oxalic acid or tartaric acid. In some embodiments, the ligand is a dendritic molecule, e.g., a metal chelating or metal binding dendritic molecule. In certain embodiments, the ligand is not a chelating agent.

In one embodiment, capturing agent comprises at least one anion selected from the group consisting of a carbonate, a phosphate, a sulfate, a silicate, a citrate, an oxalate, and combinations thereof.

In one embodiment, the capturing agent comprises at least one anion which is a soft base. Exemplary soft bases include, but are not limited to, R₂S, RSH, I⁻, SCN⁻, S₂O₃²⁻, R₃P, (RO)₃P, CN⁻, RNC, CO, C₂H₄, C₆H₆, H⁻, and R⁻, wherein R is alkyl.

In another embodiment, the capturing agent comprises at least one anion which is a hard base. Exemplary hard bases include, but are not limited to, H₂O, OH⁻, F⁻, CH₃CO₂⁻, PO₄³⁻, SO₄²⁻, CL⁻, CO₃²⁻, ClO₄^{-,} NO₃⁻, ROH, RO⁻, R₂O, NH₃, RNH₂, and N₂H₂, wherein R is alkyl.

The skilled artisan is well aware that soft Lewis bases prefer soft Lewis acids and hard Lewis bases prefer hard Lewis acids. The hard and soft acids and bases (HSAB) principle was proposed by Pearson in 1963, and has been widely used to understand chemical reactions (Pearson R G. Journal of chemical education 1987; 64(7):561-567). Accordingly, a soft or hard Lewis acid can be captured using a capturing agent comprising a soft or hard Lewis base anion as appropriate. A capturing agent's efficiency to capture or bind with an irritant, such as metal, may be decreased by using the soft Lewis base anions for capturing hard Lewis acids or using hard Lewis base anions for capturing soft Lewis acids. For example, nickel is a soft acid (Pearson R G. J Am Chem Soc 1963, 85(22):3533-3539) which has a high affinity toward soft bases (Stone F G A, & West R. Advances in organometallic chemistry: Academic Press, New York; 1979).

The capturing agent may comprise two or more different anions. Thus in certain embodiments, the capturing agent comprises at least two different anions. When two or more different anions are present, the anions may all be soft bases, all hard bases or a combination thereof. Accordingly, capturing ability of a capturing agent can be adjusted by varying the ratio of the different anions present. For example, the capturing ability of a capturing agent can be adjusted by varying the ratio of hard base anions and soft base anions. Preferably the anions are selected from the group consisting of a carbonate, a phosphate, a sulfate, a silicate, a citrate and an oxalate.

When at least two different anions are present, they may be present in an equal ratio by moles or by weight or one can be present in excess of the others, e.g. at least about 1%, at least about 5%, at least about 10%, at least about 15%, at least about 20%, at least about 50% excess to at least one of the other anions present. Percent excess can be based on moles or on weight.

In one preferred embodiment, capturing agent comprises at least one anion selected from the group consisting of a carbonate, a phosphate and a sulfate.

A number of cations can be present in the minerals. Exemplary cations include cations of alkali metals and alkali earth metals.

In one embodiment, the capturing agent comprises at least one cation selected from the group consisting of alkali metal cations and alkali earth metal cations.

In one embodiment, the capturing agent comprises at least two different cations selected from the group consisting of alkali metal cations and alkali earth metal cations.

In one embodiment, the capturing agent comprises at least one alkali metal cation and at least one alkali earth metal.

In one embodiment, the alkali metal or the alkali earth metal cation is selected from the group consisting of Li⁺, Na⁺, K⁺, Be²⁺, Mb²⁺, Ca²⁺, Sr²⁺ and Ba²⁺. Preferably, the alkali earth metal cation is Ca²⁺.

In one embodiment, the capturing agent is selected from the group consisting of calcium carbonate, calcium phosphate, apatitie such as hydroxyapatite (Ca₁₀(PO₄)₆(OH)₂), ammonium calcium silicate, sodium alumniosilicate, calcium silicate, sodium calcium aluminosilicate, magnesium silicate, tricalcium silicate, potassium bisulfite, potassium metabisulfite, sodium bisulfite, sodium metabisulfite, sodium sulfite, ferric orthophosphate, ferric phosphate, ferric pyrophosphate, ferric sodium pyrophosphate, magnesium sulfate, magnesium phosphate, manganese sulfate, manganese oxide, manganese carbonate, aluminum potassium sulfate, aluminum sodium sulfate, sodium aluminum phosphate, sodium bicarbonate, ammonium carbonate, ammonium sulfate, ammonium phosphate, and combinations thereof.

Preferably calcium phosphate is amorphous calcium phosphate, monosodium calcium phosphate, disodium calcium phosphate, trisodium calcium phosphate, tetrasodium calcium phosphate or calcium nanopowder. As used herein, the term “nanopowder” refers to a powder whose mean diameter is so small that its physical properties are substantially affected by size related confinement effects. Nanopowders usually have a mean diameter less than or equal to 250 nm, and preferably have a mean diameter less than or equal to 100 nm. More preferably, nanopowders may have a mean diameter less than 50 nm.

In one embodiment, the capturing agent is selected from materials from the Generally Recognized as Safe (GRAS) list maintained by the United Stated Food and Drug Administration.

In certain embodiments, it is desirable, but not necessary that the capturing agent particles do not detract from the tactile attributes of the finished product.

In one embodiment, the capturing agent particles have one dimension in the range of 10,000-100,000 nm, in the range of 1000-10,000 nm, in the range of 1-3000 nm, in the range of 1-1000 nm, in the range of 20-750 nm, in the range of 20-500 nm, in the range of 25-500 nm, in the range of 50-500 nm, in the range of 70-500 nm, in the range of 100-500 nm, or in the range of 100-250 nm. In some embodiments, the capturing agent particles have one dimension of about 20 nm, of about 50 nm, of about 70 nm, of about 250 nm, of about 300 nm, or of about 500 nm. Skilled artisan knows that nanoparticles that are more than 20 nm do not effectively penetrate skin in the absence of permeabilization enhancers. See, for example, Kuo et al., Biomaterials 2009.

In some embodiments, the surface area of the capturing agent particle is in the range of 0.5 m²/g to 10000 m²/g. In some embodiments, the surface area of the capturing agent particle is in the range of 250 m²/g to 10000 m²/g. In some embodiments, the surface area of the capturing agent particle is in the range of 0.5 m²/g to 1000 m²/g. In some embodiments, the surface area of the capturing agent particle is in the range of 0.5 m²/g to 500 m²/g. In some embodiments, the surface area of the capturing agent particle is in the range of 250 m²/g to 500 m²/g. In some embodiments, surface are of the capturing agent particle is in the range of 300 m²/g to 400 m²/g. It is to be understood that surface area comprises the surface area of any cavities present in the particles. The surface area can be determined by the BET techniques as described in ISO 9277 Standard.

As used here, the term “in the range of” includes the expressed or specified boundaries given for the range.

Without wishing to be bound by theory, it is postulated that the negatively charged surface of the capturing agent can bind positively charged irritant ions in an efficient manner. The surface charge of the capturing agent can be measured from c-potential (zeta-potential).

In one embodiment, the capturing agent has a negative c-potential, preferably between −1 to −20 mV, more preferably between −5 to −15 mV and most preferably between −7 to −13 mV.

In some embodiments, 100 mg of capturing agent captures 25% of 0.02M of a metal in 15, 30, 45, 60, 75, 100, 120, 180, 240, 260, or 300 minutes.

In some embodiments, 100 mg of capturing agent captures 50% of 0.02M of a metal in 15, 30, 45, 60, 75, 100, 120, 180, 240, 260, or 300 minutes. Preferably, 100 mg of capturing agent captures 50% of 0.02M nickel in 240 minutes.

In some embodiments, 100 mg of capturing agent captures 100% of 0.02M of a metal in 15, 30, 45, 60, 75, 100, 120, 180, 240, 260, or 300 minutes. Preferably, 100 mg of capturing agent captures 100% of 0.02M nickel in 260 minutes.

There are numerous suitable vehicles for facilitating the delivery of capturing agents to the tissue or surface of an irritant releasing object. A suitable vehicle is any material that can encounter the tissue or the surface of the irritant releasing object, to deliver the capturing agent to the tissue or said surface. Examples of suitable vehicles include, but are not limited to, anhydrous formulations, aqueous solutions, lotion, creams, pastes, aerosols, and the like. The capturing agent can also be applied in finely divided form as mixture with a dusting powder, e.g., as a mixture with a talcum powder or a finely divided starch powder.

In one embodiment, the capturing agent is incorporated into a pharmaceutically acceptable skin coating material that is applied to the skin. As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In one embodiment, the composition comprises, in addition to the capturing agent, at least one emollient. As used herein, the term “emollient” refers to compound which soften, lubricate and moisturize the skin as well as sooth irritation to the skin and mucous membranes, i.e., they are soothing to the skin.

In one embodiment, the emollient is selected from the group consisting of glycerine, sorbitol, fatty alcohols, hydrocarbons, triglycerides, waxes, esters, silicone oils, lanolins, and the like as well as mixtures thereof.

In one embodiment, the emollient is glycerine.

In one embodiment, the emollient is a fatty alcohol, e.g., a C₁₀₋₁₈ alcohol selected from the group consisting of decyl alcohol, lauryl alcohol, myristyl alcohol, cetyl alcohol, palmitoleyl alcohol, octyldodecanol, stearyl alcohol, oleyl alcohol and ricinoleyl alcohol.

In one embodiment, the emollient is a hydrocarbon selected from the group consisting of mineral oil, petrolatum, paraffin, squalene, polybutene, polyisobutene, hydrogenated polyisobutene, cerisin and polyethylene.

In one embodiment, the emollient is a triglyceride selected from the group consisting of castor oil, caprylic/capric triglyceride, vegetable oil, hydrogenated vegetable oil, almond oil, wheat germ oil, sesame oil, cottonseed oil, hydrogenated cottonseed oil, coconut oil, wheat germ glycerides, avocado oil, corn oil, trilaurin, hydrogenated castor oil, shea butter, cocoa butter, soybean oil, mink oil, sunflower oil, safflower oil, macadamia nut oil, olive oil, apricot kernel oil, hazelnut oil and borage oil.

In one embodiment, the emollient is a wax selected from the group consisting of carnauba wax, beeswax, candelilla wax paraffin, Japan wax, microcrystalline wax, jojoba oil, cetyl esters wax, and synthetic jojoba oil.

In one embodiment, the emollient is an ester selected from the group consisting of isopropyl myristate, isopropyl palmitate, octyl palmitate, isopropyl linoleate, C₁₂₋₁₅ alcohol benzoates, cetyl palmitate, myristyl myristate, myristyl lactate, cetyl acetate, butyl stearate, diglycol laurate, propylene glycol dicaprylate/caprate, decyl oleate, stearyl heptanoate, diisostearyl malate, octyl hydroxystearate and isopropyl isostearate.

In one embodiment, the emollient is a silicone oil selected from the group consisting of dimethicone (dimethyl polysiloxane) and cyclomethicone.

In one embodiment, the emollient is a lanolin selected from the group consisting of lanolin oil, isopropyl lanolate, acetylated lanolin alcohol, acetylated lanolin, hydroxylated lanolin, hydrogenated lanolin and lanolin wax.

The composition may also include emulsifying surfactants. Exemplary emulsifying surfactant include, but are not limited to, sorbitan monooleate, sorbitan sesquioleate, sorbitan trioleate, glyceryl stearate, sorbitan stearate, sorbitan tristearate, and the like, as well as mixtures thereof.

The compositions may also include viscosity enhancers. Viscosity enhancer include, but are not limited to, the following materials: the group consisting of polyolefin resins, polyolefin polymers, ethylene/vinyl acetate copolymers, polyethylene, and the like, as well as mixtures thereof. The composition should have a viscosity sufficient to permit easy spreading on the tissue, or on the surface of an object to be coated, and yet retain the capturing agent in a generally intact layer over the tissue or the coated object.

Humectants may also be included in the composition to provide skin moisturization benefit. Humectants are typically cosmetic ingredients used to increase the water content of the top layer of the skin. Humectants include primarily hydroscopic ingredients. Suitable humectants include, but are not limited to, Acetamide MEA, Aloe Vera Gel, Arginine PCA, Chitosan PCA, Copper PCA, Corn Glycerides, Dimethyl Imidazolinone, Fructose, Gluccamine, Glucose, Glucose Glutamate, Glucuronic Acid, Glutamic Acid, Glycereth-7, Glycereth-12, Glycereth-20, Glycereth-26, Glycerin, Honey, Hydrogentated Honey, Hydrogenated Starch Hydrolysate, Hydrolyzed Corn Starch, Lactamide MEA, Lactic Acid, Lactose Lysine PCA, Mannitol, Methyl Gluceth-10, Methy Gluceth-20, PCA, PEG-2 Lactamide, PEG-10 Propylene Glycol, Propylene Glycol Citrate, Saccharide Hydrolysate, Saccharide Isomerate, Sodium Asparate, Sodium Lactate, Sodium PCA, Sorbitol, TEZ-Lactate, TEA-PCA, Urea, xylitol, and the like, as well as mixtures thereof.

It will be apparent to those skilled in the art that additional agents may be desirable for inclusion in the present compositions. Examples include, but are not limited to, acceptable carriers, anti-inflammatories, antimicrobials, antipuretics, skin protectants, buffering agents, alpha-hydroxy acid, microbial or algal extracts and/or fractions thereof, enzyme inhibitors, antihistamines, antioxidants, analgesics, astringents, fragrances, dyes, natural and/or synthetic virtamin analogs, sunscreens, deodorants, and combinations thereof.

In certain embodiments, the composition of the invention further comprises an immuno-suppressing agent. The term “immuno-suppressing agent” refers to a compound which possesses immune response inhibitory activity. Examples of immuno-suppressing agents include, but are not limited to, corticosteroids, cyclosporin A, FK506, rapamycin, leflunomide, deoxyspergualin, prednisone, azathioprine, mycophenolate mofetil, OKT3, ATAG, interferon and mizoribine. In certain embodiments, the immuno-suppressing agent is a corticosteroid. Preferably, the immuno-suppressing agent is a corticosteroid selected from the group consisting of clobetasol propionate, clobetasone butyrate, hydrocortisone, hydrocortisone acetate, fluocinolone acetonide and mometasone furoate.

In certain embodiments, the composition of the invention is in the form of an emulsion. The term “emulsion,” as used herein, includes both classic oil-in water dispersion or droplets, as well as other lipid structures which can form as a result of hydrophobic forces which drive apolar residues (i.e., long hydrocarbon chains) away from water and polar head groups toward water, when a water immiscible oily phase is mixed with an aqueous phase. These other lipid structures include, but are not limited to, unilamellar, paucilamellar, and multilamellar lipid vesicles, micelles, and lamellar phases.

In certain embodiments, the composition of the invention is in the form of an injectable formulation. Preferably, the compositions comprise an acceptable vehicle for an injectable formulation. This vehicle can be, in particular, a sterile, isotonic saline solution (monosodium or disodium phosphate, sodium, potassium, calcium or magnesium chloride, and the like, or mixtures of such salts), or dry, in particular lyophilized, compositions which, on addition, as appropriate, of sterilized water or of physiological saline, enable injectable solutions to be formed The preferred sterile injectable preparations can be a solution or suspension in a nontoxic parenterally acceptable solvent or diluent.

In some embodiments, the capturing agent comprises from about 0.001% to about 99%, from about 0.001% to about 50%, from about 0.001% to about 40%, from about 0.001% to about 30%, from about 0.001% to about 20%, from about 0.001% to about 10%, from about 0.001% to about 5%, from about 0.01% to about 20%, or from about 1% to about 25%, from about 20% of the total weight of the composition.

In one embodiment, one or more components of the composition described herein can act as a buffer to prevent or limit a galvanic reaction which may occur in the presence of sweat.

In another aspect, the invention provides for a method of inhibiting/preventing metal exposure to tissue, the method comprising applying to the tissue a composition described herein. As used herein, the term “applying” refers to the placement of a composition onto the surface of a tissue, e.g. skin.

The amount of the capturing agent to be applied is generally an therapeutically effective amount. The phrase “therapeutically-effective amount” as used herein means that amount of capturing agent which is effective for producing some desired therapeutic effect, e.g., inhibiting an immunological event on contact of an irritant with a tissue, at a reasonable benefit/risk ratio applicable to any medical treatment.

Determination of a therapeutically effective amount is well within the capability of those skilled in the art. Generally, a therapeutically effective amount can vary with type and amount of irritant, condition of the tissue surface, and administration of other pharmaceutically active agents. Generally, the capturing agent is applied in the range of about 0.01 pg per square centimeter to about 100 pg per square centimeter of the tissue surface area.

In certain embodiments, the composition is applied topically to the tissue. The compositions may be applied to the tissue as a cream, lotion or moisturizer, through a spray, a wipe, cotton wrap, bandage, or any combinations thereof.

In certain embodiments, the composition can be applied to an article of clothing, e.g., before wearing of the clothing article. It is to be understood that the composition is applied to the clothing surface that comes in contact with skin. For example, the composition can be applied to inside surface of gloves.

In other embodiments, the composition is administered via an oral rinse solution.

In one embodiment, the composition is applied to the tissue before initiating contact with the irritant.

In one embodiment, the tissue is skin, preferably mammal skin, e.g. human or animal skin.

The composition of the invention can be applied to the tissue and then allowed to capture irritants, e.g. metal, and then rinsed away. The rinsing can be done with regular tap-water, mineral water, distilled water or phosphate buffered saline. In one embodiment, the method comprises capture of metals present on surface of a tissue by a composition described herein and rinsing to remove metal bound capturing agents of the composition.

In another embodiment, the composition is applied to the skin and then removed by normal desquamatory events (normal sloughing of outer most layer of skin) and/or personal hygiene.

In one embodiment, the composition is applied once per day, or once every 2 or 3 days. In another embodiment, the composition only needs to be applied once every 2-4 weeks or less often.

In one embodiment, the composition is applied after personal hygiene, e.g., hand washing, and/or before/after physical activity, e.g. activity involving contact with an irritant.

In another aspect, the invention provides for a method of inhibiting metal release from an object, the method comprising coating said object with a composition described herein. As used herein, the term “coating” is subgenerically defined to include thin films, thick films and thicker structures. Without wishing to be bound by theory, inhibition of metal release from an object reduces/inhibits the metal induced contact dermatitis potential of the object. The object can be coated with the composition by methods well known in the art for coating objects. Exemplary methods include, spraying, printing, using brushes, wiping, by bare hands, and/or dipping the object to be coated in a solution comprising the composition to be used for coating. After application of the composition, the coated object can be dried.

In one embodiment, the object is selected from the group consisting of jewelry, coins, zippers, snaps, eyeglasses, electronic devices, wristwatches and toys.

In one embodiment, the object is submersed into a solution containing a composition described herein.

In one embodiment, the coating on the surface of said object does not alter the appearance of said object, e.g. the appearance of metal does not change.

The actual amount of capturing agent that can be applied to the tissue or the surface of the contact dermatitis causing object, will vary, and can be routinely determined given the present disclosure depending upon the type of capturing agent and amount of irritant. Different capturing agents will have disparate capacities for binding various irritants and, accordingly, more or less will be required depending on the choice of capturing agent used. However, it is critical that enough is used to inhibit or reduce the contact dermatitis caused by the irritant. Typically, the amount of capturing agent applied will be in the range of about 0.01 pg per square centimeter to about 100 pg per square centimeter.

In another aspect, the invention provides for a method of preparing a metal exposure reducing composition, the method comprising blending a capturing agent in an emollient, cream, lotion or moisturizer.

In a further aspect, the invention provides for a method of inhibiting/preventing metal exposure to tissue, the method comprising, blending a capturing agent in an emollient, cream, lotion or moisturizer and applying on site of interest before coming into contact with an object capable of inducing metal contact dermitatis.

In addition to prevention of contact dermatitis, results provide a platform for removal or complex formation of nickel or other metals ions. This can be useful as a potential replacement of EDTA or other chelating agents to potentially strip metal ions, e.g. nickel ions, from a resin or other substrate to free bound proteins.

The compositions described herein may also be used to reduce concentration of nickel or other metal ions for applications that may include a treatment regime to reduce metal ion toxicity (i.e. passing nickel containing blood through a column of calcium carbonate particles).

The compositions described here in can also be used to inhibit activity of enzymes that are dependent on metal cofactors. Without wishing to be bound, capture of cofactor metal with a composition described herein inhibits the activity of the enzyme. In one embodiment, the invention provides for a method of inhibiting activity of matrix metalloproteinase (MMP), the method comprising capturing zinc with a composition described herein.

### Definitions

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Other than in the operating examples, or where otherwise indicated, all numbers expressing quantities of ingredients or reaction conditions used herein should be understood as modified in all instances by the term “about.” The term “about” when used in connection with percentages may mean ±1%.

The singular terms “a,” “an,” and “the” include plural referents unless context clearly indicates otherwise. Similarly, the word “or” is intended to include “and” unless the context clearly indicates otherwise.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

The present invention may be defined in any of the following numbered paragraphs:
1. A composition for inhibiting irritant exposure to a tissue, wherein the composition comprises a non-covalently crosslinked capturing agent and where the capturing agent cannot transverse through the tissue.
2. A composition for inhibiting irritant exposure to tissue, wherein the composition comprises a nanoparticle comprising a capturing agent.
3. The composition of paragraph 2, wherein the composition further comprises an encapsulation agent, where the encapsulation agent encapsulates the nanoparticle.
4. The composition of paragraph 3, wherein the encapsulation agent is a polymer or a hydrogel.
5. The composition of any of paragraphs 2-4, wherein the nanoparticle is a nanoshell, nanocage, core-shell particle, nano-rod, nano-wire, nano-cube, hollow nanosphere, aggregate of nanoparticles, or a combination thereof.
6. The composition of any of paragraphs 2-5, wherein the capturing agent is the nanoparticle.
7. A composition for inhibiting irritant exposure to tissue, wherein the composition comprises a capturing agent that has a surface area greater than 1, 10, 20, 50 m²/g.
8. A composition for inhibiting irritant exposure to tissue, wherein the composition comprises a capturing agent that releases calcium ions upon binding to the metal.
9. A composition for inhibiting metal exposure to tissue, wherein the composition comprises an insoluble capturing agent having a water content greater than 10%, 50%, 75%.
10. The composition of paragraph 9, wherein the capturing agent comprises a chelating agent.
11. The composition of any of paragraphs 1-9, wherein the capturing agent is an inorganic capturing agent.
12. The composition of paragraph any of paragraphs 1-9, wherein the capturing agent is a crystalline compound.
13. The composition of paragraph any of paragraphs 1-12, wherein the capturing agent comprises both crosslinked and non-crosslinked components.
14. The composition of any of paragraphs 1-13, wherein the capturing agent is not an ion exchange resin
15. The composition of any of paragraphs 1-14, wherein the capturing agent is not derivatized with a chelating agent.
16. The composition of any of paragraphs 1-15, wherein the capture agent comprises both inorganic and organic components.
17. The composition of any of paragraphs 1-16, wherein the capturing agent is a chelating agent.
18. The composition of any of paragraphs 1-17, wherein the capturing agent is an adsorbent agent.
19. The composition of any of paragraphs 1-18, wherein the composition is an emulsion.
20. The composition of any of paragraphs 1-19, wherein the composition is an injectable formulation.
21. The composition of any of paragraphs 1-20, wherein the capturing agent is a particle.
22. The composition of paragraph 21, wherein the particle is non-symmetrical, irregular, spherical, rod-like, elongated or star-shaped.
23. The composition of paragraph 21, wherein the particle is a nanoparticle or a microparticle.
24. The composition of paragraph 21, wherein the particle is not derivatized with a chelating agent.
25. The composition of paragraph 21, wherein the particle is a degradable particle.
26. The composition of paragraph 21, wherein the particle is a Janus particle.
27. The composition of paragraph 21, wherein said particle comprises at least one of a silicate, a carbonate, a sulfate, a phosphate, a citrate, an oxalate, or a combination thereof.
28. The composition of paragraph 21, wherein said particle comprises at least one of an alkali metal and/or alkali earth metal.
29. The composition of any of paragraphs 1-28, wherein said capturing agent is selected from the group consisting of calcium carbonate, calcium phosphate, hydroxyapatite, ammonium calcium silicate, sodium alumniosilicate, calcium silicate, sodium calcium aluminosilicate, magnesium silicate, tricalcium silicate, potassium bisulfite, potassium metabisulfite, sodium bisulfite, sodium metabisulfite, sodium sulfite, ferric orthophosphate, ferric phosphate, ferric pyrophosphate, ferric sodium pyrophosphate, magnesium sulfate, magnesium phosphate, manganese sulfate, manganese oxide, manganese carbonate, aluminum potassium sulfate, aluminum sodium sulfate, sodium aluminum phosphate, sodium bicarbonate, ammonium carbonate, ammonium sulfate, ammonium phosphate, and combinations thereof.
30. The composition paragraph 29, wherein said calcium phosphate is amorphous calcium phosphate, monosodium calcium phosphate, disodium calcium phosphate, trisodium calcium phosphate, tetrasodium calcium phosphate or calcium nanopowder.
31. The composition of paragraph 21, wherein the particle is a porous particle.
32. The composition of paragraph 21, wherein the particle has size 1-3000 nm.
33. The composition of paragraph 21, wherein said particle has a negative or neutral zeta potential.
34. The composition of paragraph 21, wherein said particle has a surface area between 0.5 m²/g and less than 1000 m²/g.
35. The composition of any of paragraphs 1-34, wherein said composition has between 0.001-99% by weight of said capturing agent.
36. The composition of any of paragraphs 1-35, wherein said tissue is skin or mucosa.
37. The composition of any of paragraphs 1-36, wherein said irritant is a metal selected from the group consisting of soft Lewis acids or hard Lewis acids.
38. The composition of paragraph 37, wherein said soft Lewis acid is at least one of Cu⁺, Ag⁺, Au⁺, Tl⁺Hg⁺, Cs⁺, Zn²⁺, Ni²⁺, Pd²⁺, Cd²⁺, Pt²⁺, Hg²⁺, Tl³⁺, or metal atoms with zero oxidation state.
39. The composition of paragraph 37, wherein said hard Lewis acid is at least one of Cr³⁺, Co³⁺, Fe³⁺, La³⁺, In³⁺, Ga³⁺, Sr³⁺, Al³⁺, or metal atoms with zero oxidation state.
40. The composition of any of paragraphs 1-39, further comprising at least one emollient.
41. The composition of paragraph 40, wherein said emollient is selected from the group consisting of glycerine, sorbitol, fatty alcohol, ethylene glycol, hydrocarbon, triglyceride, wax, ester, silicone oil, vegetable oil and lanolin.
42. The composition of paragraph 41, wherein said fatty alcohol is a C₁₀₋₁₈ alcohol selected from the group consisting of decyl alcohol, lauryl alcohol, myristyl alcohol, cetyl alcohol, palmitoleyl alcohol, octyldodecanol, stearyl alcohol, oleyl alcohol and ricinoleyl alcohol.
43. The composition of paragraph 41, wherein said hydrocarbon is selected from the group consisting of mineral oil, petrolatum, paraffin, squalene, polybutene, polyisobutene, hydrogenated polyisobutene, cerisin and polyethylene.
44. The composition of paragraph 41, wherein said triglyceride is selected from the group consisting of castor oil, caprylic/capric triglyceride, vegetable oil, hydrogenated vegetable oil, almond oil, wheat germ oil, sesame oil, cottonseed oil, hydrogenated cottonseed oil, coconut oil, wheat germ glycerides, avocado oil, corn oil, trilaurin, hydrogenated castor oil, shea butter, cocoa butter, soybean oil, mink oil, sunflower oil, safflower oil, macadamia nut oil, olive oil, apricot kernel oil, hazelnut oil and borage oil.
45. The composition of paragraph 41, wherein said wax is selected from the group consisting of carnauba wax, beeswax, candelilla wax paraffin, Japan wax, microcrystalline wax, jojoba oil, cetyl esters wax, and synthetic jojoba oil.
46. The composition of paragraph 41, wherein said ester is selected from the group consisting of isopropyl myristate, isopropyl palmitate, octyl palmitate, isopropyl linoleate, C₁₂₋₁₅ alcohol benzoates, cetyl palmitate, myristyl myristate, myristyl lactate, cetyl acetate, butyl stearate, diglycol laurate, propylene glycol dicaprylate/caprate, decyl oleate, stearyl heptanoate, diisostearyl malate, octyl hydroxystearate and isopropyl isostearate.
47. The composition of paragraph 41, wherein said silicone oil is selected from the group consisting of dimethicone (dimethyl polysiloxane) and cyclomethicone.
48. The composition of paragraph 41, wherein said lanolin is chosen from the group consisting of lanolin oil, isopropyl lanolate, acetylated lanolin alcohol, acetylated lanolin, hydroxylated lanolin, hydrogenated lanolin and lanolin wax.
49. The composition of any of paragraphs 1-48, further comprising at least one immuno-suppressing agent.
50. The composition of paragraph 49, wherein the immuno-suppressing agent is a corticosteroid.
51. The composition of paragraph 50, wherein the corticosteroid is selected from the group consisting of clobetasol propionate, clobetasone butyrate, hydrocortisone, hydrocortisone acetate, fluocinolone acetonide and mometasone furoate.
52. The composition of paragraph 9, wherein the insoluble capturing agent is a polymer or a hydrogel.
53. The composition of paragraph 21, wherein the core of the particle comprises a non-zero concentration of a chelating agent.
54. A method of inhibiting irritant exposure to tissue, the method comprising applying to the tissue a composition of any of paragraphs 1-53 to the tissue.
55. A method of inhibiting irritant exposure to tissue, the method comprising applying to the tissue a composition comprising a nanoparticle, wherein the nanoparticle has a surface area between 0.5 m²/g and 1000 m²/g.
56. A method of preventing irritant exposure to a tissue, the method comprising the steps of:
   a) capturing metals on surface of a tissue with a composition of any of paragraphs 1-53; and
   b) rinsing to remove metal bound composition.
57. The method of paragraph 56, wherein said rinsing is with regular tap-water, mineral water, distilled water or phosphate buffered saline.
58. A method of inhibiting irritant exposure to tissue, the method comprising the steps of:
   a) blending a composition of any one of paragraphs 1-53 in an emollient, cream, lotion or moisturizer; and
   b) applying on site of interest before coming into contact with an object capable of stimulating irritant allergy.
59. The method of paragraph 58, wherein 0.1-99% by weight of capturing agent is blended with said emollient, cream, lotion or moisturizer.
60. The method of paragraph 54, 58 or 59, wherein said composition is applied to the tissue before initiating contact with an object capable of stimulating irritant allergy.
61. The method of paragraph 60, wherein said tissue is skin or mucosa.
62. The method of paragraph 60, wherein the composition is applied topically, or administered via oral rinse solution.
63. The method of paragraph 62, wherein the composition is applied to the tissue through a spray, a wipe, cotton wrap, bandage or glove.
64. A method of inhibiting irritant release from an object, the method comprising coating the object with a composition of any one of paragraphs 1-53.
65. A method of paragraph 64, wherein the object is coated by spraying, printing, bare hand or dipping into the composition followed by drying.
66. A method of capturing irritants using appropriate combination of soft-soft acid/base or soft-hard acid/bases.
67. A method of capturing nickel using a composition of any one of paragraphs 1-53, wherein 100 mg of the capturing agent captures 50% of 0.02M nickel in 240 minutes.
68. A method of capturing nickel using a composition of any one of paragraphs 1-53, wherein 100 mg of the capturing agent captures 100% of 0.02M nickel in 260 minutes.
69. A method of decreasing inorganic capturing agent's efficiency to capture a metal, the method comprising using a composition of any one of paragraphs 1-53, wherein the composition comprises a soft Lewis base to capture a hard Lewis acid and/or a hard Lewis base to capture a soft Lewis acid.
70. A method of inhibiting activity of matrix metalloproteinase (MMP) in a tissue of interest, the method comprising capturing zinc with a composition of any of paragraphs 1-53.
71. The method of paragraph 70, wherein the capturing agent can infiltrate the tissue.
72. A method for removing at least one metal as an insoluble compound, the method comprising applying a composition of any one of paragraphs 1-53 to a tissue of interest.

To the extent not already indicated, it will be understood by those of ordinary skill in the art that any one of the various embodiments herein described and illustrated can be further modified to incorporate features shown in any of the other embodiments disclosed herein.

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### EXAMPLES

### Materials and Methods

Materials: CaCO₃-particles were purchased from Specialty Mineral Inc, MA, USA. Nickel, palladium, cadmium and cobalt wires were purchased from Puratronic®, and standardized artificial eccrine perspiration solution (artificial sweat (20% v/v in all cases unless otherwise specified), pH 6.1) was purchased from Pickering Laboratories, CA, USA, and glycerin was purchased from Walgreens. Calcium phosphate particles and hydroxyapatite nano-powder were purchased from Sigma Aldrich (St. Louis, Mo.). All reagents were used as-received unless otherwise mentioned.

Skin preparation: Pig full-thickness samples were obtained from the back and flank of female Yorkshire pigs. Excess hair was removed from the skin using electric hair clippers. The skin was then harvested within 1 hour after the animal was sacrificed. After the subcutaneous fat was removed from the skin using razor blades, the skin was sectioned into stripes and stored at (−80° C.) for up to 12 months. Before use in following experiments, the skin was thawed for an hour by immersing in phosphate buffer saline.

Preparation of CaCO₃ or CaP-particles coated nickel wire: 0.5 g of CaCO₃ or CaP-particles were suspended in double distilled water, vortexed for 15 mins at room temperature and nickel wires (2 cm length, 0.5 mm diameter, 40 mg weight) were immersed for 10 min. Subsequently wires were removed and washed twice with double distilled water and air dried.

Quantification of nickel release from nickel wires: Nanoparticle coated nickel wires were incubated in 2 ml of artificial sweat, at regular time points (1, 2, 3 and 4 days) 100 μL of solution was diluted 1000 times with 2% v/v HNO₃ (Sigma Aldrich (St. Louis, Mo.) aqueous solution, and subjected to Inductively Coupled Plasma Atomic Emission Spectrophotometer (ICP-AES, Horiba Jobin Yvon, Activa S) to measure concentration of nickel.

Quantification of metal sequestered by the nanoparticles: Two sets of experiments were performed to quantify the amount of nickel (either from NiSO₄ solution or released from nickel wire) by CaCO₃ or CaP nanoparticles. Set 1. Nanoparticles (0.5 g) were suspended in 0.2 M of NiSO₄ aqueous solution, after incubation of 4 hr, particles were centrifuged (20,000 rpm for 15 min) and the supernatant was collected and subjected to ICP-AES. Set 2. 50 mg of particles were dispersed in artificial sweat; subsequently metal wires (2 cm length, 0.5 mm diameter, 40 mg weight) were submersed. After 48 hrs the concentration of metal in the supernatant was measured using ICP-AES. In a positive control experiment, uncoated metal wires were immersed in artificial sweat, and after 48 hrs concentration of metal was quantified using ICP-AES.

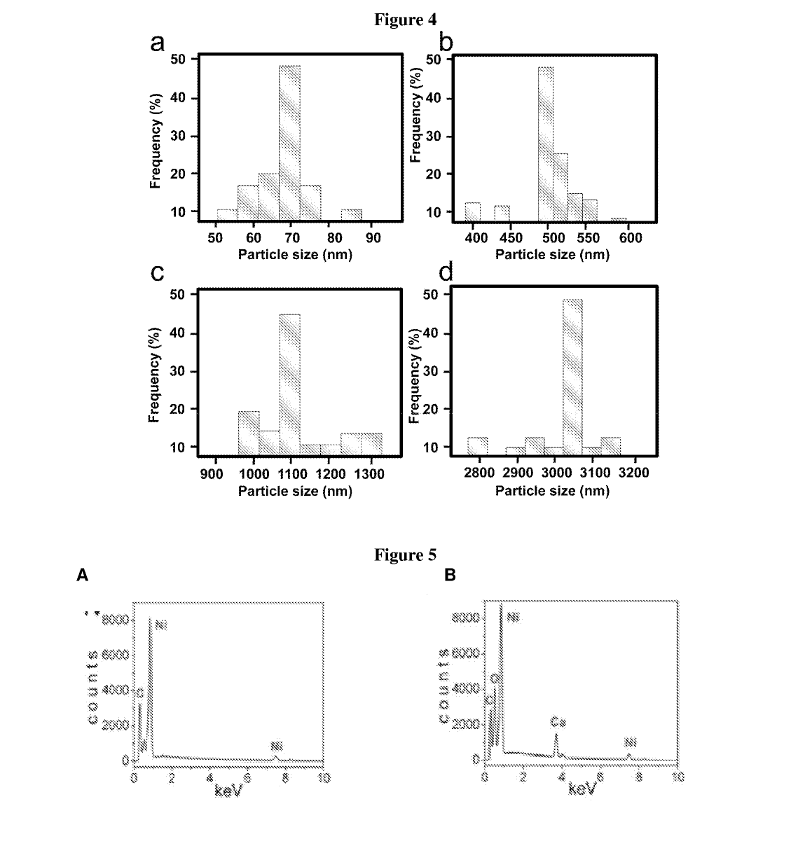

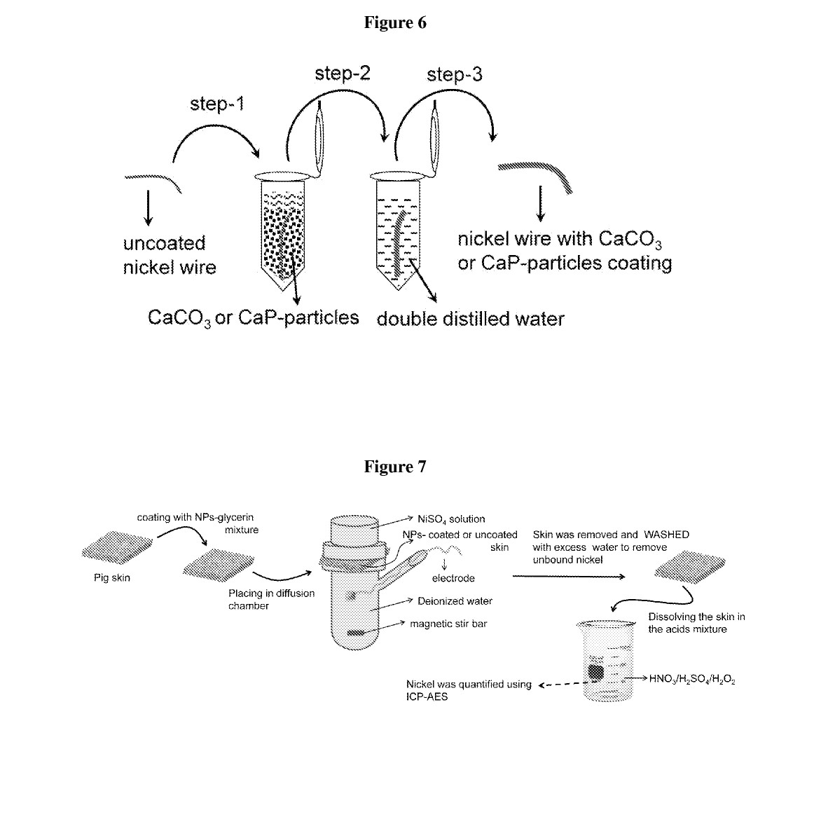

SEM and EDX analysis: Skin samples for SEM/EDX were prepared as follows. Full-thickness pig skin was cut into 2×2 cm with surgical blade, and placed in a Petri dish. Artificial sweat (300 μL) was added on top of skin, and after 10 min nanoparticles dispersed in glycerin (50 μL) was applied with a spatula to make a thin layer. After 30 min, 50 μL of NiSO₄ (3 mM) solution was added and the skin was then vertically sectioned (5 mm thickness) with a surgical blade and placed on an aluminum stub with carbon tape (for visualization see FIG. 4a). Similarly, after 5 hrs, surface of the skin was washed with deionized water to remove the nanoparticle-glycerin coating followed by vertical sectioning. The samples were examined using Environmental SEM (FEI/Phillips XL30 FEG-ESEM) operated at 10 kV. EDX and elemental mapping analysis data was also collected from the same samples at 10 kV using X-ray detector (from TSL) coupled with FEI/Phillips XL30 FEG-ESEM (base was rotated at 45° to image vertical sections). Using similar procedure only glycerin coated and uncoated skin samples were prepared.

XPS analysis: CaP or CaCO₃-particles were incubated with NiSO₄ solution overnight then isolated by centrifugation and air dried; a thin film of NPs was prepared on copper tape by simple adhesion, subsequently XPS analysis was performed by a Kratos AXIS Ultra Imaging X-ray photoelectron spectrophotometer equipped with a monochromatized Al K_{α} source. The spectrometer was configured to operate at high resolution with pass energy of 15 eV. NiSO₄ salt was been used as control.

Surface area measurements: Specific surface area analysis was performed using the Brunauer-Emmett-Teller (BET) method of nitrogen gas adsorption/desorption as described by Volodkin D V, Larionova N I, & Sukhorukov G B. Protein encapsulation via porous CaCO3 microparticles templating. Biomacromolecules 2004, 5(5):1962-72.

### Example 1

### Nickel Chelating Potential of Soft Bases

Nickel chelating potential of soft bases including phosphate and carbonate ions was examined first. In general, soft acids/bases have less charge (lower charge state) and larger radius, whereas hard acids/bases have a high charge and smaller radius. The inventors demonstrated the efficiency of CaCO₃ and CaP-particles to bind to free nickel ions that are released from a metal source and thus reduce exposure to skin.

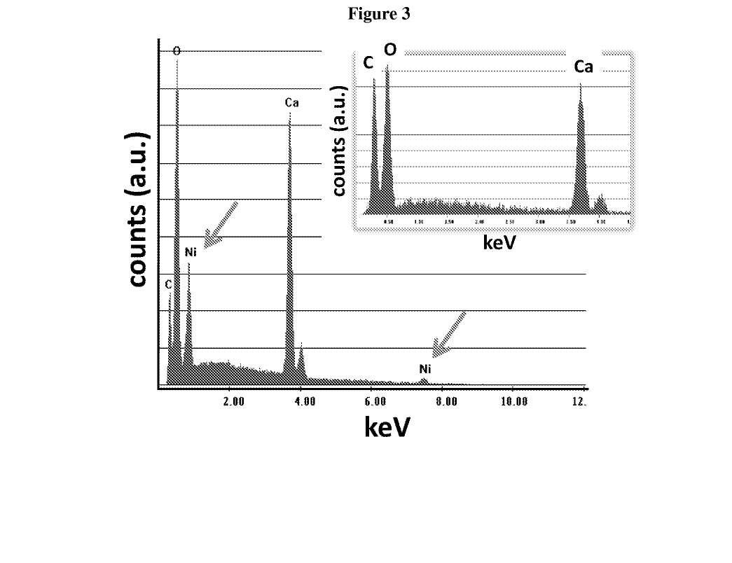

The inventors first examined the ability of CaCO₃ or CaP-particles to bind to nickel ions that are released from either a nickel salt or a nickel-wire in a solution. Either CaCO₃ or CaP-particles (0.5 g) were suspended in NiSO₄ aqueous solution (0.2 M, solution was made with 20% (v/v) artificial sweat (containing minerals, metabolites, and 20 amino acids), pH 6.1), after incubation for 48 hr, the nickel concentration in the supernatant was measured using Inductively Coupled Plasma Atomic Emission Spectrometer (ICP-AES). Use of artificial sweat is an industry standard for testing the release of metal ions such as nickel from jewelry. See, for example, Midander, et al., Nickel release from nickel particles in artificial sweat. Contact Dermatitis 2007; 56(6):325-30. The data summarized in Table 1a (entry 1) shows that CaCO₃ and CaP-particles efficiently capture nickel and significantly reduce the nickel concentration (>99%). In addition, energy-dispersive X-ray (EDX) analysis of these particles showed the characteristic peaks corresponding to calcium and nickel at 3.6 keV (Ca-Kα), 0.85 (Ni-Lα) and 7.47 (Ni-Kα), which confirmed the particles' ability to sequester nickel (FIG. 3). Subsequently, the ability of the nanoparticles to capture nickel ions that are continually released from a metal source was evaluated by immersing nickel wires in a suspension of CaCO₃ or CaP-particles. The suspended particles were shown to effectively capture the nickel ions released from nickel wire as evidenced after 72 hr in the presence of artificial sweat by ICP-AES analysis (Table 1a, entry 2). In addition, CaCO₃ and CaP-particles were seen to capture other metal ions that can provoke an allergy such as palladium, cadmium and cobalt (Table 1a, entry 3-5).

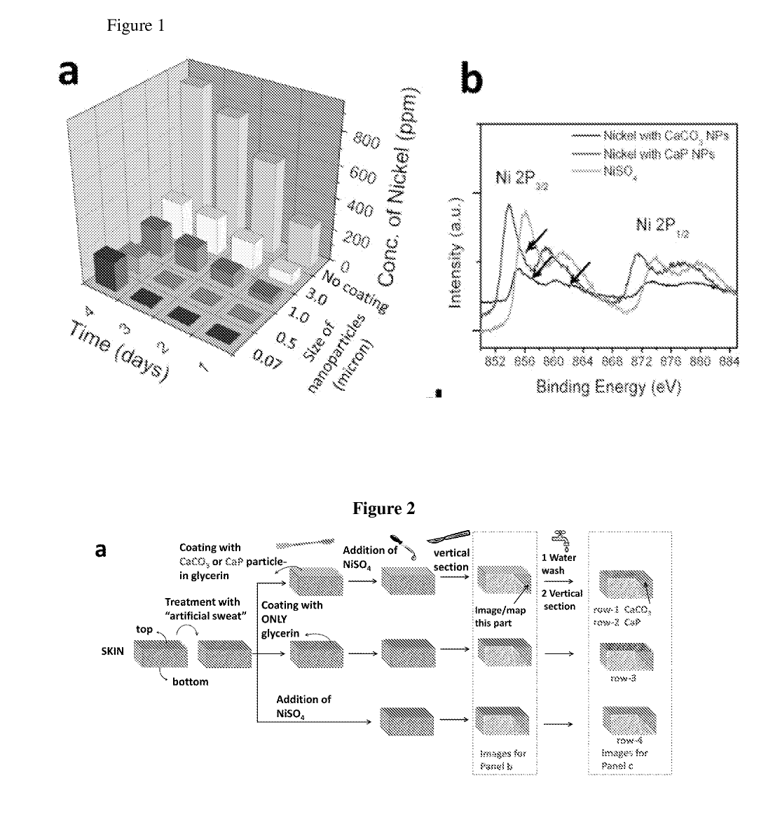

The inventors discovered that particles under ˜500 nm are the most efficient nickel chelators (Table 1b and FIG. 1a) during nickel sequester experiments using different sizes of CaCO₃-particles (equal amounts of 70, 500, 1000 and 3000 nm (for particle size distribution from dynamic light scattering analysis see FIG. 4)). As expected, the surface area increased as size of the particles decreased (Table 1b). Thus, the high surface area of the smaller particles is likely responsible for the enhanced efficacy. Likewise, coatings containing smaller particles more effectively captured nickel that was released from nickel containing objects (FIG. 1a). Particles under ˜500 nm that were directly coated onto the nickel-wire most effectively captured nickel ions that were released from the metal source (FIG. 1a) as observed via ICP AES analysis. The particle based coating not only captured the nickel released from the wire but prevented the nickel ions from further becoming liberated into solution.

To investigate the nature of nickel binding to the particles, after scavenging nickel from NiSO₄ solution, isolated CaCO₃ and CaP-particles were analyzed by X-ray photoelectron spectroscopy (XPS). The Ni 2P core level spectrum (FIG. 1b) was resolved into a two spin-orbit pair (with splitting of ˜18 eV) which is in agreement with values reported by Matienzo et al. (Inorganic Chemistry 1973, 12(12):2762-2769). The Ni 2P₃₁₂ binding energies (BE) were 856.1, 854.8 and 854.2 eV for NiSO₄, nickel-captured CaP and nickel-captured CaCO₃-particles, respectively (FIG. 1b). The BE of nickel-captured CaCO₃-particles (854.2) matched with the reported BE of NiCO₃ reported by Matienzo et al. (Inorganic Chemistry 1973, 12(12):2762-2769), indicating the formation of NiCO₃ as nickel was sequestered by the CaCO₃-particles. Similarly, the BE of nickel captured CaP-particles matched with reported NiPO₄ suggesting the chelation of nickel with phosphates (Practical Surface Analysis: Auger and X-Ray Photoelectron Spectroscopy: Wiley, New York; 1990). The potential existence of physically adsorbed nickel is possible; however, it likely represents only a small percentage of bound nickel given the presence of small shoulder peaks in the XPS spectra (FIG. 1b shown in arrows). The inventors have further extended this approach to capture other soft acid metal ions including palladium and cadmium, and a hard acid metal ion such as cobalt (Table 1a). Intriguingly, a significant reduction in chelation efficiency (˜40% less) was observed for the hard acid cobalt ions (Table 1a, entry 5) compared to the soft acid nickel (Table 1a, entry 2). Despite the reduced surface area of the CaCO₃-particles compared to CaP-particles (Table 1a), as anticipated by the HSAB principle, CaCO₃-particles exhibited 10% higher efficiency than CaP-particles to capture cobalt ions. Thus, the nature of the metal and the capturing agent (soft or hard acid/base) impacts the efficiency of the sequestering process.

Generally, adsorption of ions onto a salt does not typically promote counterion release. Accordingly, without wishing to be bound by a theory, direct chelation of nickel with either CaP or CaCO₃-particles can trigger the release of calcium. To study this, after incubation of particles in deionized water for 24 hr with either nickel or zinc ions (0.01 M), the concentration of calcium in the solution was measured (Table 2). Indeed, in the presence of nickel or zinc a ˜10-fold excess of calcium ions were released, which indicates cation exchange during chelation.

### Example 2

### Nanoparticles Prevent Penetration of Nickel Ions into Tissue

The inventors next evaluated the ability of CaCO3 or CaP-particles to prevent penetration of nickel ions into tissue; nanoparticles were dispersed in glycerin (an emollient), and applied as thin layer on top of pig skin (FIG. 2a). The size of the nanoparticles used was 70-2000 nm for the CaCO₃ particles and 100-2000 nm for the CaP particles. SEM showed the presence of nanoparticles coating on the skin (data not shown). Subsequently, a high concentration (3 mM) of NiSO₄ solution was added on top of the skin and incubated for 5 hrs. The solution was not permitted to contact the underside of the skin. The skin samples were either vertically sectioned and examined for elemental mapping (SEM-EDX) to visualize the location of nickel, or washed with deionized water, vertically sectioned, and subjected to SEM-EDX to visualize the location and amount of nickel that remained within the skin (i.e. analogous to determining the impact of hand washing or showering). For these experiments, non-coated and emollient (glycerin) coated skin samples were used as controls. The presence of a coating containing 20% (w/w) CaCO₃ or CaP-particles in glycerin significantly reduced skin exposure to nickel ions (data not shown). For the experimental groups containing nanoparticles no detectable nickel was seen to permeate through the skin. Furthermore, the particles were retained on the surface of the skin (data not shown) and could easily be removed with water without any residue from nickel or the nanoparticles (data not shown). On the contrary, for the glycerin only coated samples and uncoated samples, the elemental mapping images showed that high concentrations of nickel permeated through the skin (data not shown).

To quantify the efficiency of nanoparticles to prevent nickel penetration into the skin; either CaCO₃ or CaP-particles (20% w/w)) in glycerin was applied on the full-thickness pig skin and placed in vertical Franz diffusion cells. The receiver cell was filled with deionized water, and the donor cell was filled with a NiSO₄ (0.05 M) solution (FIG. 7). Uncoated skin and glycerin coated skin samples were used as controls. After 48 hr skin was removed from the diffusion chamber and rinsed multiple times with deionized water to remove unbound and particle-bound nickel. Subsequently, skin was dissolved using nitric acid/sulfuric acid/hydrogen peroxide mixture and the nickel concentration was quantified using ICP-AES. As shown in Table 3, bare skin and glycerin coated skin retained higher amounts of nickel ions while coatings containing either CaCO₃ or CaP-particles prevented nickel ion penetration. Overall, these results show that GRAS-based nanoparticles applied as a coating to the skin within a simple emollient significantly reduce exposure to metal ions such as nickel.

Above discussed results show GRAS-based nanoparticles that contain soft base ions are proficient for the prophylaxis of soft acid metal ions. The size of the nanoparticles plays a pertinent role in efficacy; smaller particles (<500 nm) are efficient to bind nickel compared to those in the 1-3 micron size range due to large surface area of smaller particles, and carbonates/phosphates capture nickel more efficiently than cobalt due to their higher affinity towards soft acid nickel than hard acid cobalt. Thus, use of GRAS-based nanoparticles within topical compositions represents an effective and safe approach to limit the exposure to metal ions, which should be beneficial both occupationally and socially to the tens of millions of people throughout the world who suffer from metal induced contact dermatitis.

TABLE 1
Ability of CaCO₃ and CaP particles to bind nickel and other metals.
(a) Tabular form of metal concentration change upon incubation with
nanoparticles in the solution (measured using ICP-AES). Source of
metal was either metal salt (entry 1) or metal-wire (entry 2-5).
The last two columns on the right depict the percentage of metal that
was captured by the nanoparticles. Size of the nanoparticles used was
70-2000 and 100-2000 nm for CaCO₃ and CaP-particles, respectively,
(b) Equal amounts (0.5 g) of CaCO₃-particles (70, 500,
1000, 3000 nm) were combined with NiSO₄ (25000 ppm in
artificial sweat) after 24 hr, particles were removed and the concentration
of nickel in the supernatant was measured using ICP-AES. In all cases,
values are average of three independent experiments and all standard
deviations were <5% of the average values.
a

| | | Conc. of metal (ppm) | | | | |
|---|---|---|---|---|---|---|
| | | WITHOUT | WITH particles | | % of metal decrease | |
| entry | metal | particles | ^{a}CaCO₃ | ^{b}CaP | CaCO₃ | CaP |
| 1 | NiSO₄ | 12301.0 | 73.8 | 24.6 | 99.4 | 99.8 |
| 2 | Ni-wire | 786.5 | 7.0 | 3.1 | 99.1 | 99.6 |
| 3 | Pd-wire | 629.3 | 133.4 | 118.9 | 78.8 | 81.1 |
| 4 | Cd-wire | 1108.5 | 185.7 | 112.4 | 83.2 | 89.6 |
| 5 | Co-wire | 926.0 | 352.8 | 419.4 | 61.9 | 54.7 |

b

| | Size of CaCO₃ | Surface area | Conc. of Nickel | % of Ni |
|---|---|---|---|---|
| entry | Particles (μm) | (m²/g) | (ppm) | decrease |
| 1 | without particles | — | 25000 | 0.0 |
| 2 | 0.07 | 28.686 | 2475 | 90.1 |
| 3 | 0.5 | 20.674 | 5412 | 78.3 |
| 4 | 1 | 10.900 | 7675 | 69.3 |
| 5 | 3 | 7.732 | 8418 | 66.2 |

^{a}Surface area is 20.67 m²/g
^{b}Surface area is 52.95 m²/g

TABLE 2
Release of calcium ions from calcium-based particles. Tabular form
of calcium ion concentration that was released from CaCO₃ and CaP-
particles in the presence and absence of metal ions such as nickel
and zinc. Particles were suspended in deionized water, after 24 hr
incubation with either nickel or zinc ions, the concentration of calcium
in the solution was measured using ICP-AES. Indeed, in the presence
of nickel or zinc, an excess of calcium ions was released which suggests
cation exchange during chelation. Size of the nanoparticles used was
70-2000 and 100-2000 nm for CaCO₃ and CaP-particles, respectively.
In all cases, values are average of three independent experiments
and all standard deviations were <5% of the average values.

| | | Additional | Conc. of |
|---|---|---|---|
| entry | Nanoparticles | metal ion | calcium (ppm) |
| 1. | CaCO₃ | — | 3.8 |
| 2. | CaCO₃ | nickel | 31.2 |
| 3. | CaCO₃ | zinc | 20.3 |
| 4. | Ca—P | — | 4.2 |
| 5. | Ca—P | nickel | 43.0 |
| 6. | Ca—P | zinc | 27.4 |

TABLE 3
Prophylaxis efficiency of nanoparticle coating to prevent nickel ion
penetration into skin. CaCO₃ or CaP-particles in glycerin was applied
to pig skin, placed into a diffusion chamber and subsequently exposed to
nickel ions. After 48 hr, skin was removed from the chamber and rinsed
multiple times with deionized water to remove excess unbound and
particles-bound nickel ions. Subsequently, skin was dissolved in
1:1mixture of HNO₃ and H₂SO₄ and subjected to H₂O₂. The nickel
concentration in the solution was quantified using ICP-AES. Size of the
nanoparticles is 70-2000 and 100-2000 nm for CaCO₃ and CaP-particles,
respectively. In all cases, values are average of three independent
experiments and all standard deviations were <5% of the average values.

| Coating on | Conc. of Nickel |
|---|---|
| pig skin | (ppm) |
| uncoated | 407.2 |
| glycerin | 421.1 |
| (emollient) | |
| CaCO₃-glycerin | 2.9 |
| CaP-glycerin | 1.7 |

### REFERENCES

1. Marks J G, Jr., Belsito D V, DeLeo V A, Fowler J F, Jr., Fransway A F, Maibach H I, Mathias C G, Pratt M D, Rietschel R L, Sherertz E F and others. North American Contact Dermatitis Group patch-test results, 1996-1998. Arch Dermatol 2000; 136(2):272-3.
2. Rietschel R L, Fowler J F, Warshaw E M, Belsito D, DeLeo V A, Maibach H I, Marks J G, Mathias C G, Pratt M, Sasseville D and others. Detection of nickel sensitivity has increased in North American patch-test patients. Dermatitis 2008; 19(1):16-9.
3. Jacob SE, Moennich J N, McKean B A, Zirwas M J, Taylor J S. Nickel allergy in the United States: a public health issue in need of a “nickel directive”. J Am Acad Dermatol 2009; 60(6):1067-9.
4. Hidaka S, Okamoto Y, Abe K. Elutions of metal ions from dental casting alloys and their effect on calcium phosphate precipitation and transformation. J Biomed Mater Res 1994; 28(2):175-80.
5. Dou X, Liu L L, Zhu X J. Nickel-elicited systemic contact dermatitis. Contact Dermatitis 2003; 48(3):126-9.
6. Raison-Peyron N, Guillard O, Khalil Z, Guilhou J J, Guillot B. Nickel-elicited systemic contact dermatitis from a peripheral intravenous catheter. Contact Dermatitis 2005; 53(4):222-5.
7. Nosbaum A, Rival-Tringali A L, Barth X, Damon H, Vital-Durand D, Claudy A, Faure M. Nickel-induced systemic allergic dermatitis from a sacral neurostimulator. Contact Dermatitis 2008; 59(5):319-20.
8. Luo J, Bercovitch L. Cellphone contact dermatitis with nickel allergy. Cmaj 2008; 178(1):23-4.
9. Suneja T, Flanagan K H, Glaser D A. Blue-jean button nickel: prevalence and prevention of its release from buttons. Dermatitis 2007; 18(4):208-11.
10. Heim K E, McKean B A. Children's clothing fasteners as a potential source of exposure to releasable nickel ions. Contact Dermatitis 2009; 60(2):100-5.
11. Nestle F O, Speidel H, Speidel M O. Metallurgy: high nickel release from 1- and 2-euro coins. Nature 2002; 419(6903):132.
12. Staton I, Ma R, Evans N, Hutchinson R W, McLeod C W, Gawkrodger D J. Dermal nickel exposure associated with coin handling and in various occupational settings: assessment using a newly developed finger immersion method. Br J Dermatol 2006; 154(4):658-64.
13. Bruckner A L, Weston W L, Morelli J G. Does sensitization to contact allergens begin in infancy? Pediatrics 2000; 105(1):e3.
14. Memon A A, Molokhia M M, Friedmann P S. The inhibitory effects of topical chelating agents and antioxidants on nickel-induced hypersensitivity reactions. J Am Acad Dermatol 1994; 30(4):560-5.
15. Menne T, Kaaber K. Treatment of pompholyx due to nickel allergy with chelating agents. Contact Dermatitis 1978; 4(5):289-90.
16. Wohrl S, Kriechbaumer N, Hemmer W, Focke M, Brannath W, Gotz M, Jarisch R. A cream containing the chelator DTPA (diethylenetriaminepenta-acetic acid) can prevent contact allergic reactions to metals. Contact Dermatitis 2001; 44(4):224-8.
17. Pantini G, Forestieri R, Brunetta F, Bencini P L. Preliminary Communication: Treatment of irritant contact dermatitis in workmen with perfluoropolymethylisopropyl ether (Fomblin H C). Int J Cosmet Sci 1990; 12(6):273-9.
18. Kaaber K, Menne T, Tjell J C, Veien N. Antabuse treatment of nickel dermatitis. Chelation—a new principle in the treatment of nickel dermatitis. Contact Dermatitis 1979; 5(4):221-8.
19. Martindale W. The Extra Pharmacopoeia: Pharmaceutical Press; 1993.
20. Christensen O B, Kristensen M. Treatment with disulfiram in chronic nickel hand dermatitis. Contact Dermatitis 1982; 8(1):59-63.
21. Hopfer S M, Linden J V, Rezuke W N, O'Brien J E, Smith L, Watters F, Sunderman F W, Jr. Increased nickel concentrations in body fluids of patients with chronic alcoholism during disulfiram therapy. Res Commun Chem Pathol Pharmacol 1987; 55(1):101-9.
22. Gawkrodger D J, Healy J, Howe A M. The prevention of nickel contact dermatitis. A review of the use of binding agents and barrier creams. Contact Dermatitis 1995; 32(5):257-65.
23. Kauffman R E, Banner J W, Blumer J L, Gorman R L, Lambert G H, Snodgrass W. Clioquinol (iodochlorohydroxyquin, Vioform) and iodoquinol (diiodohydroxyquin): blindness and neuropathy. Pediatrics 1990; 86(5):797-798.
24. White B R, Stackhouse B T, Holcombe J A. Magnetic gamma-Fe(2)O(3) nanoparticles coated with poly-1-cysteine for chelation of As(III), Cu(II), Cd(II), Ni(II), Pb(II) and Zn(II). J Hazard Mater 2009; 161(2-3):848-53.
25. Huhtinen P, Kivela M, Kuronen O, Hagren V, Takalo H, Tenhu H, Lovgren T, Harma H. Synthesis, characterization, and application of Eu(III), Tb(III), Sm(III), and Dy(III) lanthanide chelate nanoparticle labels. Anal Chem 2005; 77(8):2643-8.
26. Kuo T R, Wu C L, Hsu C T, Lo W, Chiang S J, Lin S J, Dong C Y, Chen C C. Chemical enhancer induced changes in the mechanisms of transdermal delivery of zinc oxide nanoparticles. Biomaterials 2009.
27. Pearson R G. Hard and soft acids and bases. J Am Chem Soc 1963; 85(22):3533-3539.
28. Stone F G A, West R. Advances in organometallic chemistry: Academic Press, New York; 1979.
29. Pearson R G. Recent advances in the concept of hard and soft acids and bases. Journal of chemical education 1987; 64(7):561-567.
30. Midander K, Pan J, Wallinder T O, Heim K, Leygraf C. Nickel release from nickel particles in artificial sweat. Contact Dermatitis 2007; 56(6):325-30.
31. Matienzo L J, Yin L I, Grim S O, Swartz Jr W E. X-ray photoelectron spectroscopy of nickel compounds. Inorganic Chemistry 1973; 12(12):2762-2769.
32. Practical Surface Analysis: Auger and X-Ray Photoelectron Spectroscopy: Wiley, New York; 1990.
33. Volodkin D V, Larionova N I, Sukhorukov G B. Protein encapsulation via porous CaCO3 microparticles templating. Biomacromolecules 2004; 5(5):1962-72.
   Content of all patents and other publications identified and listed in the specification is herein incorporated by reference in its entirety.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A composition for inhibiting exposure of an agent to a tissue or inhibiting contact dermatitis, wherein the composition comprises:
(i) a non-covalently cross-linked capturing agent and wherein the capturing agent cannot transverse through the tissue;
(ii) a nanoparticle comprising a capturing agent; or
(iii) a capturing agent and wherein the capturing agent is a particle.

2. (canceled)

3. (canceled)

4. (canceled)

5. (canceled)

6. (canceled)

7. The composition of claim 1, wherein the capturing agent has a surface area greater than 1, 10, 20, 50 m²/g; or the capturing agent releases cations ions upon binding the agent; or the capturing agent is an insoluble capturing agent.

8. (canceled)

9. (canceled)

10. The composition of claim 1, wherein the capturing agent comprises a chelating agent or the capturing agent is an inorganic capturing agent which is a non-symmetrical, irregular, spherical, rod-like, elongated or star-shaped particle.

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. The composition of claim 1, wherein the particle is a nanoparticle or a microparticle.

24. (canceled)

25. (canceled)

26. (canceled)

27. The composition of claim 1, wherein said particle comprises at least one of a silicate, a carbonate, a sulfate, a phosphate, a citrate, an oxalate, or a combination thereof.

28. The composition of claim 1, wherein said particle comprises at least one of an alkali metal and/or alkali earth metal.

29. The composition of claim 1, wherein said capturing agent is selected from the group consisting of calcium carbonate, calcium phosphate, hydroxyapatite, ammonium calcium silicate, sodium alumniosilicate, calcium silicate, sodium calcium aluminosilicate, magnesium silicate, tricalcium silicate, potassium bisulfite, potassium metabisulfite, sodium bisulfite, sodium metabisulfite, sodium sulfite, ferric orthophosphate, ferric phosphate, ferric pyrophosphate, ferric sodium pyrophosphate, magnesium sulfate, magnesium phosphate, manganese sulfate, manganese oxide, manganese carbonate, aluminum potassium sulfate, aluminum sodium sulfate, sodium aluminum phosphate, sodium bicarbonate, ammonium carbonate, ammonium sulfate, ammonium phosphate, and combinations thereof, wherein the calcium phosphate is amorphous calcium phosphate, monosodium calcium phosphate, disodium calcium phosphate, trisodium calcium phosphate, tetrasodium calcium phosphate or calcium nanopowder.

30. (canceled)

31. (canceled)

32. The composition of claim 1, wherein the particle has size 20 nm-100,000 nm.

33. (canceled)

34. (canceled)

35. (canceled)

36. (canceled)

37. The composition of claim 1, wherein said agent is selected from the group consisting of metals, enzymes, proteases, lipases, glycosidases, latex, grass, weeds, trees, animal products, bacterial by-products, cytokines, eicosanoids, bile acids, endotoxins, superantigens, poison ivy, and dust.

38. (canceled)

39. (canceled)

40. The composition of claim 1, wherein the composition further comprises at least one emollient.

41. (canceled)

42. (canceled)

43. (canceled)

44. (canceled)

45. (canceled)

46. (canceled)

47. (canceled)

48. (canceled)

49. The composition of claim 1, further comprising an agent selected from the group consisting of immuno-suppressing agents, anti-inflammatories, antimicrobials, antipuretics, skin protectants, buffering agents, alpha-hydroxy acid, microbial or algal extracts, fractions of microbial or algal extracts, enzyme inhibitors, antihistamines, antioxidants, analgesics, astringents, fragrances, dyes, natural or synthetic vitamin analogs, sunscreens, deodorants, and any combination thereof.

50. (canceled)

51. (canceled)

52. (canceled)

53. (canceled)

54. A method of inhibiting contact dermatitis inducing agent exposure to tissue, the method comprising applying to the tissue, an object comprising the contact dermatitis inducing agent, or an article of clothing, a composition of claim 1.

55. (canceled)

56. (canceled)

57. (canceled)

58. (canceled)

59. (canceled)

60. (canceled)

61. (canceled)

62. The method of claim 54, wherein the composition is applied topically, administered via oral rinse solution, through a spray, a wipe, cotton wrap, bandage or glove.

63. (canceled)

64. A method of inhibiting release of a contact dermatitis inducing agent from an object, the method comprising coating the object with a composition of claim 1.

65. (canceled)

66. (canceled)

67. (canceled)

68. (canceled)

69. (canceled)

70. A method of inhibiting activity of an enzyme in a tissue, the method comprising applying a composition of claim 1 to the tissue.

71. (canceled)

72. (canceled)
